(19) United States
(12) Patent Application Publication
(10) Pub. No.: US 2013/0123824 A1
(43) Pub. Date: May 16, 2013

(54) LANCING DEVICE WITH CAM-ACTUATED DRIVE AND SEPARATE GUIDANCE

(71) Applicant: FACET TECHNOLOGIES, LLC, Kennesaw, GA (US)

(72) Inventor: Douglas A. VINE, Alpharetta, GA (US)

(73) Assignee: FACET TECHNOLOGIES, LLC, Kennesaw, GA (US)

(21) Appl. No.: 13/666,377

(22) Filed: Nov. 1, 2012

Related U.S. Application Data
(60) Provisional application No. 61/556,897, filed on Nov. 8, 2011.

(51) Int. Cl. A61B 5/151

(52) U.S. Cl. CPC A61B 5/15117
USPC 606/181

(57) ABSTRACT

A lancet assembly comprises a housing and a needle carriage movably housed within the housing, the needle carriage adapted to support a needle thereon, the needle carriage having a first end and a second end generally opposite thereto. A needle is mounted to the needle carriage and a guide element is positioned within the housing for guiding the needle carriage for reciprocating translation. A pivotal drive cam is provided for driving the needle carriage in translation and has a cam lobe for engaging the second end of the needle carriage, wherein the needle carriage is guided by the guide element and driven in translation by the pivotal drive cam such that the guidance and drive are kept separate. The pivotal drive cam can be moved between a cocked, retracted position, a fired, retracted position, and an extended position in between the cocked retracted position and the fired, retracted position. The lancet assembly can include a guidance biasing spring for biasing the needle carriage toward a retracted position and a separate drive spring for storing potential energy for pivoting the pivotal drive cam.

### CROSS-REFERENCE TO RELATED APPLICATION

This application claims the benefit of U.S. Provisional Patent Application Ser. No. 61/556,897, filed Nov. 8, 2011, the entirety of which is hereby incorporated herein by reference for all purposes.

### TECHNICAL FIELD

The present invention relates generally to the field of medical devices, and more particularly to an improved lancing device for blood testing and typing applications.

### BACKGROUND

In typical known lancets, a needle (lance) is affixed to some sort of movable needle carrier, such as a sled or carriage. A drive spring urges the needle carrier outwardly toward an extended position in which the needle extends beyond the housing to penetrate the skin or tissue of the user. Another spring is positioned for urging the needle carrier inwardly toward a retracted position in which the needle withdrawn and does not extend beyond the housing. In such a device it is common to try to balance these springs more or less to try to avoid “bouncing” of the needle and a double strike of the user by the lancing device. This balancing effort can be difficult. Typically, these springs, which tend to urge the needles toward extended and retracted positions, are acting on the same part of the lancing device (normally the carrier). Having the drive springs act on parts that are also involved in guidance tends to distort the guidance efforts, as well as the drive efforts. Thus, by having one mechanism involved in both functions, both functions are diminished somewhat.

### SUMMARY

In example embodiments, the present invention provides a lancet assembly having a housing and a needle carriage movably housed within the housing. The needle carriage is adapted to support a needle thereon, and has a first end and a second end generally opposite thereto. A needle is mounted to the needle carriage and a guide element is positioned within the housing for guiding the needle carriage for reciprocating translation. A pivotal drive cam is provided for driving the needle carriage in translation and has a cam lobe for engaging the second end of the needle carriage. The needle carriage is guided by the guide element and is driven in translation by the pivotal drive cam such that the guidance and drive functions are kept substantially separate from one another.

Preferably, the pivotal drive cam is movable between (1) a cocked, retracted position, (2) a fired, retracted position, and (3) an extended position between the cocked retracted position and the fired, retracted position.

Optionally, the lancet assembly further comprises a guidance biasing spring for biasing the needle carriage toward a retracted position and a separate drive spring for storing potential energy for pivoting the pivotal drive cam. Advantageously, these springs need not act against one another and need not be carefully balanced one against the other. Optionally, the second end of the needle carriage can include a cam follower portion for engaging the cam lobe and being driven thereby.

The needle carriage can be provided with an elongate guided surface extending generally axially between the first and second ends of the needle carriage and the guide element can include a guide surface for engaging and guiding the guided surface of the needle carriage. In one preferred form, the guide element within the housing and the guided surface of the needle carriage define a sliding dovetail connection.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1A is a side view of a lancing device with cam-actuated drive and separate guidance according to a first example embodiment of the present invention.

FIG. 1B is another side view of the lancing device with cam-actuated drive and separate guidance of FIG. 1A.

FIG. 1C is a perspective view of the lancing device with cam-actuated drive and separate guidance of FIG. 1A.

FIG. 1D is a side view of the lancing device guide surface and needle carriage according to a preferred embodiment of the present invention.

FIG. 2A is a side view of a lancing device with cam-actuated drive and separate guidance according to another example embodiment.

FIG. 2B is another side view of the lancing device with cam-actuated drive and separate guidance of FIG. 2A.

FIG. 3A is a side view of a lancing device with cam-actuated drive and separate guidance according to another example embodiment.

FIG. 3B is another side view of the lancing device with cam-actuated drive and separate guidance of FIG. 3A.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

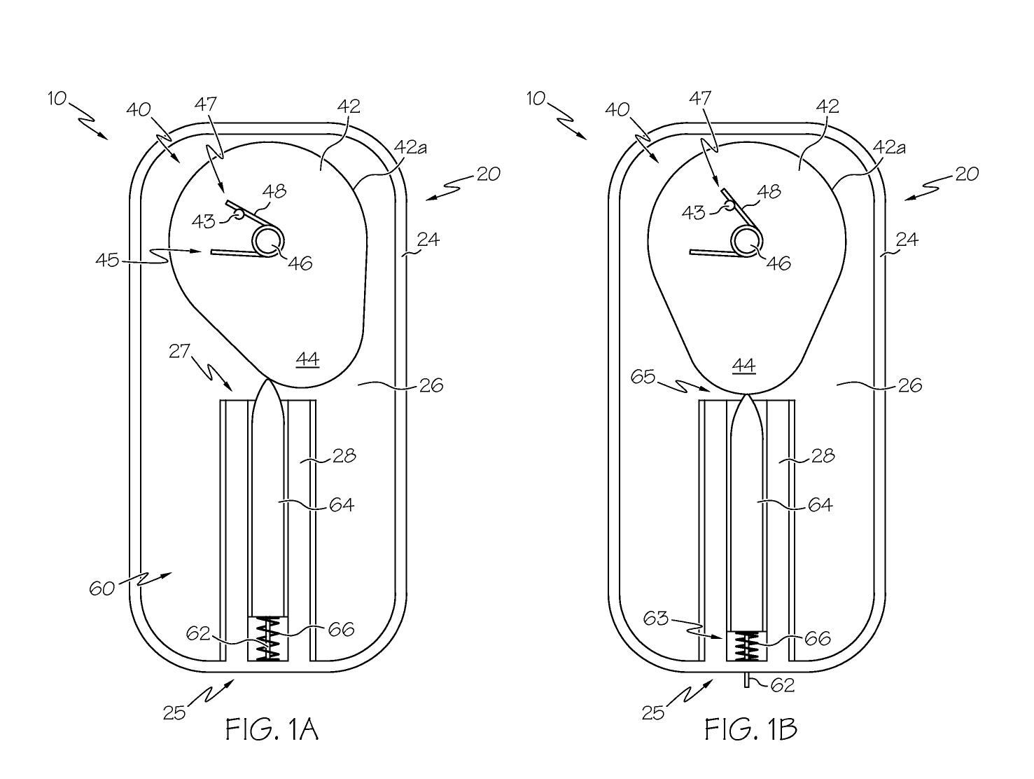

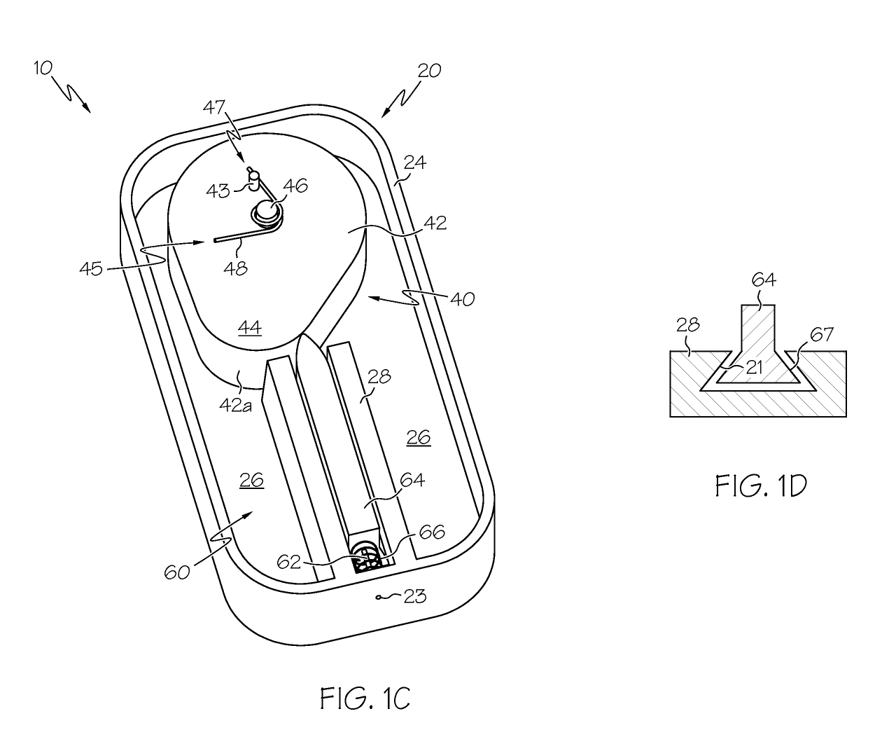

With reference now to the drawing figures, wherein like reference numbers represent corresponding parts throughout the several views, FIGS. 1A-3B depict various embodiments of lancing devices according to example embodiments of the present invention. In general, the lancing devices herein include a lancet assembly having a housing and a needle carriage movably housed within the housing, the needle carriage adapted to support a needle thereon, the needle carriage having a first end and a second end generally opposite thereto. A needle is mounted to the needle carriage and a guide element is positioned within the housing for guiding the needle carriage for reciprocating translation. A pivotal drive cam is provided for driving the needle carriage in translation and has a cam lobe for engaging the second end of the needle carriage, wherein the needle carriage is guided by the guide element and driven in translation by the pivotal drive cam such that the guidance and drive are kept separate.

FIGS. 1A-3C depict a lancing device 10 with cam-actuated drive and separate guidance according to a first example embodiment of the present invention. In general, the lancing device 10 includes a housing 20 containing a cam assembly 40 and needle assembly 60. In example embodiments, the housing 20 includes a peripheral wall 24 that stretches substantially along the perimeter edge and together with a floor defines a recessed interior portion 26. The housing also includes a guide element 28 and a needle passage 23 integrally formed therein. Optionally, the wall 24 has a substantially constant thickness and the recessed interior portion preferably maintains a thickness substantially similar or greater than the wall thickness.

The guide element 28 includes a first exterior end 25 adjacent to wall 24 and a second interior end 27 located within the recessed interior portion 26. In preferred example embodiments, a needle carriage 64, having a first end 63 and a second end 65 generally opposite thereto, is movably housed within the housing 20 and is adapted to support a needle 62. As best seen in FIG. 10, the needle carriage 64 can be provided with an elongate guided surface 67 extending generally axially between the first and second ends of the carriage and the guide element 28 can include a guide surface 21 for engaging and guiding the guided surface 67 of the needle carriage 64. In one preferred embodiment, the guide element 28 within the housing 20 and the guided surface 67 of the needle carriage 44 comprise a sliding dovetail connection). Optionally the guide element 28 is a one-piece construction, but the guide element can be provided as a multi-piece construction. Also, multiple smaller guide elements can be employed, as desired.

A pivotal drive cam 42, having an outer surface or profile 42a, is provided for driving the needle carriage in translation and includes a cam lobe 44 for engaging the second end of the needle carriage. The pivotal drive cam is pivotally mounted to a pivot on a mounting pin 46 within the interior portion 26. The needle carriage 64 is guided by the guide element 28 and driven in translation by the drive cam 42 such that the guidance and drive are kept separate from one another.

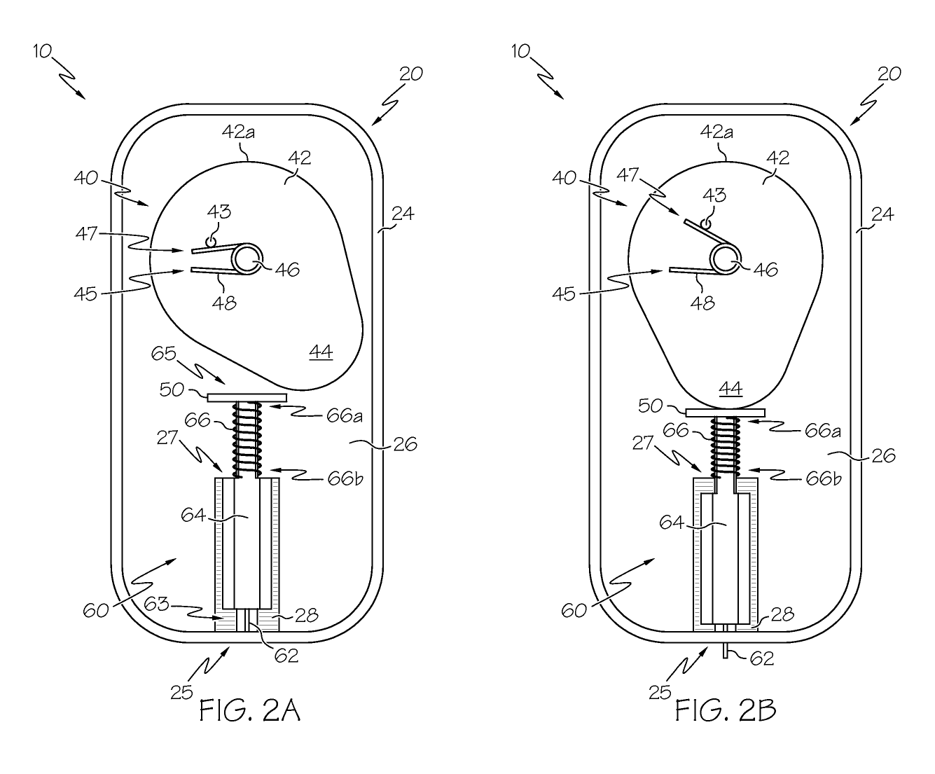

In example embodiments, the lancet device 10 can optionally include a guidance biasing spring 66 for biasing the needle carriage toward a retracted position and a separate drive spring 48 for storing potential energy for driving the pivotal drive cam 42. In preferred example embodiments, the guidance biasing spring 66, located within the guide element 28, is fixed between the first end 63 of the needle carriage 64 and guide element first end 25 (best seen in FIGS. 1A-C). The biasing spring is axially aligned with the needle carriage 64 and the needle 62. In other example embodiments, the biasing spring 66 is located at the second end 27 of the needle carriage 64. A proximal end 66a of spring 46, axially aligned with the needle carriage, mounts below a flat cam follower portion 50 that extends beyond the outer edge of the needle carriage and a distal end 66b of the spring is fixed at the second end 27 of guide element 28 (best seen in FIGS. 2A-B).

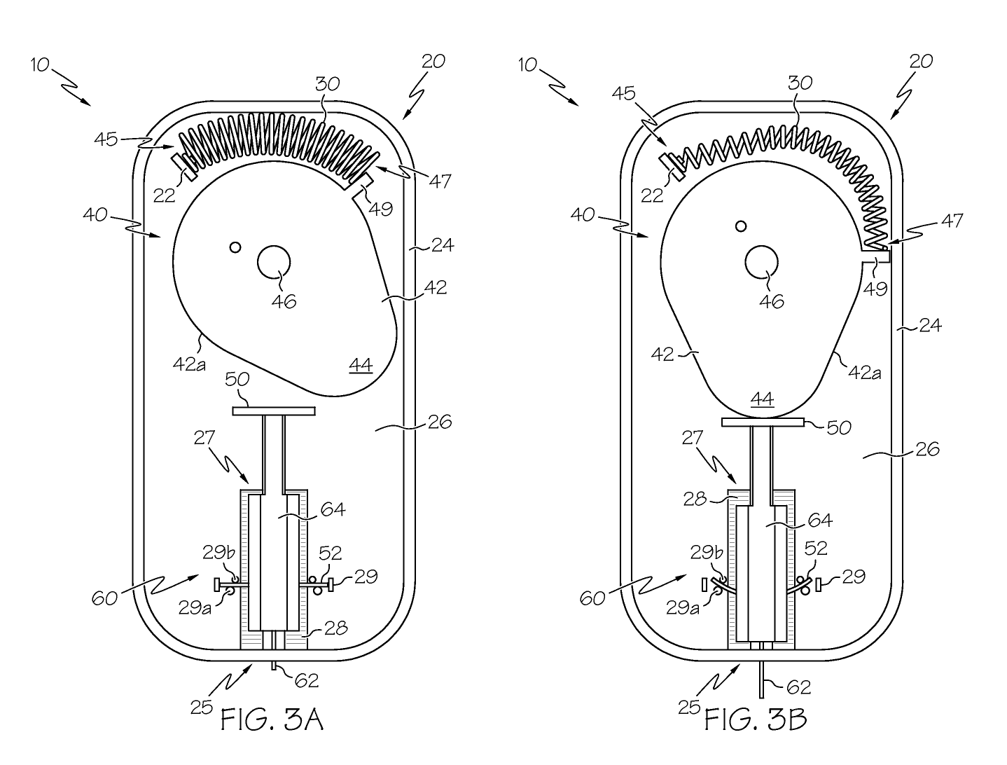

In other example embodiments, the biasing spring can comprise a leaf spring 52 fixed between the first and second ends of the guide element 28 (best seen in FIGS. 3A-B). The leaf spring sits perpendicular to the translational movement of the needle carriage 64 and is secured by side stops 29, front housing pins 29a, and rear housing pins 29b. Additionally, the bottom surface of the needle carriage 64 preferably includes a protrusion (not shown). The protrusion extends from the bottom surface of the needle carriage 64 towards recessed interior portion 26 and initiates contact with the biasing spring 52 when acted upon by the drive cam 22.

Referring again to FIG. 1 et seq., the drive spring 48 has a first arm/end 45 fixed to the interior portion 26 and a second arm/end 47 fixed to the drive cam pin 43. The drive spring 48 permits potential energy to be stored for pivoting the pivotal drive cam 42 and driving the needle carriage 64 in translation. In example embodiments, the pivotal drive cam is movable between a cocked, retracted position (best seen in FIGS. 1A, 2A, 3A), a fired, retracted position (best seen in FIGS. 1B, 2B,3B), and an extended position between the cocked retracted position and the fired, retracted position (best seen in FIG. 1C). The drive spring 48 is preferably a compression spring. In additional embodiments, the pivotal drive cam 42 can be altered to receive a compression spring. FIGS. 3A-B depict a cam assembly 40 comprising a compression spring. The compression drive spring 30, having a first end 45 fixed to the interior spring mount 22 and a second end 47 fixed to a pivotal drive cam arm 49, additionally permits storing potential energy for pivoting the pivotal drive cam 42 and driving a needle carriage 64 in translation. Alternately, other types of springs could be used.

In example embodiments, the cam lobe 44 can be designed to achieve a variety of motions for a variety of purposes. For example, the cam lobe can be designed to have a shape that minimized the time duration of the needle exposure or that minimizes the stroke to just barely jut the needle from the end of the housing. The lobe can also be designed to control the acceleration of the needle as it enters the skin (the lobe can be designed to cause the needle to be accelerating, decelerating, or maintaining a more or less constant velocity as the needle enters the skin). For example, if the shortest skin or tissue penetration is desired, the lobe could be further pointed so that there is a very small duration of time when the lobe is engaged with the needle carriage. Alternatively, if a longer skin or tissue penetration is desired, the lobe could be further rounded and extended so that there is a longer duration of time when the lobe is engaged with the needle carriage. By careful design of the cam lobe, the motion and acceleration of the needle into and out of the skin can be carefully controlled. Additionally, to further adjust the speed and acceleration of the needle, the mass of the cam and the carriage can be altered relative to one another to provide a desired force-to-mass ratio (and consequent acceleration as the cam lobe accelerates through its motion). Those skilled in the art will also appreciate that the drive cam spring strength also plays a role.

In operation, the cocking and releasing of the pivotal drive cam 42 can be performed in a variety of different ways. In preferred example embodiments, the cam 42 can have a recessed knob or button (not shown) that extends through the housing 20. The knob permits cocking the cam to a cocked, retracted position, and also includes a function to permit firing.

The components discussed and described herein can be formed from a variety of materials as desired by a user. In example embodiments, the lancet device and components can be formed from plastics (e.g., polystyrene), other polymers, glass, metals, metal alloys, resins, rubbers, rubber derivatives, elastomerics (i.e. santoprene), silicones or other known materials. In preferred embodiments, the drive cam or the needle carriage may be formed from a low friction material. Materials of such low friction properties can include nylon or PTFE. Additionally, the springs can be formed from plastics, other polymers, metals, metal alloys, resins, rubbers, rubber derivatives, or other known materials.

While the invention has been described with reference to preferred and example embodiments, it will be understood by those skilled in the art that a variety of modifications, additions and deletions are within the scope of the invention, as defined by the following claims.

What is claimed is:

1. A lancet assembly comprising;
a housing;
a needle carriage movably mounted within the housing, the needle carriage adapted to support a needle thereon, the needle carriage having a first end and a second end generally opposite thereto;
a needle mounted to the needle carriage;
a guide element positioned within the housing for guiding the needle carriage for reciprocating translation; and
a pivotal drive cam for driving the needle carriage in translation and having a cam lobe for engaging the second end of the needle carriage;
wherein the needle carriage is guided by the guide element and driven in translation by the pivotal drive cam such that guidance of the needle carriage and drive of the needle carriage are kept substantially separate from one another.

2. A lancet assembly as claimed in claim 1 wherein the pivotal drive cam is movable between a cocked, retracted position, a fired, retracted position, and an extended position between the cocked retracted position and the fired, retracted position.

3. A lancet assembly as claimed in claim 1 further comprising a guidance biasing spring for biasing the needle carriage toward a retracted position and a drive spring for storing potential energy for pivoting the pivotal drive cam.

4. A lancet assembly as claimed in claim 1 further comprising a biasing spring for biasing the needle carriage toward a retracted position and a drive spring for storing potential energy for pivoting the pivotal drive cam, and wherein these springs are separate springs from one another.

5. A lancet assembly as claimed in claim 3 wherein the biasing spring is operative for biasing the needle carriage in translation and the drive spring is operative for pivoting the pivotal drive cam.

6. A lancet assembly as claimed in claim 1 wherein the second end of the needle carriage comprises a cam follower portion for engaging the cam lobe and being driven thereby.

7. A lancet assembly as claimed in claim 1 wherein the needle carriage has an elongate guided surface extending generally axially between the first and second ends of the needle carriage and wherein the guide element includes a guide surface for engaging and guiding the guided surface of the needle carriage.

8. A lancet assembly as claimed in claim 7 wherein the guide element within the housing and the guided surface of the needle carriage define a sliding dovetail connection.

9. A lancet assembly comprising;
a housing;
a needle carriage movably mounted within the housing, the needle carriage adapted to support a needle thereon, the needle carriage having a first end and a second end generally opposite thereto;
a needle mounted to the needle carriage;
a guide element positioned within the housing for guiding the needle carriage for reciprocating translation; and
a drive mechanism for driving the needle carriage in translation;
wherein the needle carriage is guided by the guide element and driven in translation by the drive mechanism and wherein the guidance of the needle carriage and drive of the needle carriage are kept substantially separate from one another.

10. A lancet assembly as claimed in claim 9 wherein the drive mechanism comprises a pivotal drive cam movable between a cocked, retracted position, a fired, retracted position, and an extended position between the cocked retracted position and the fired, retracted position.

11. A lancet assembly as claimed in claim 9 further comprising a guidance biasing spring for biasing the needle carriage toward a retracted position and a drive spring for storing potential energy for pivoting the drive mechanism.

12. A lancet assembly as claimed in claim 9 further comprising a biasing spring for biasing the needle carriage toward a retracted position and a drive spring for storing potential energy for pivoting the drive mechanism, and wherein these springs are separate springs from one another.

13. A lancet assembly as claimed in claim 9 wherein the drive mechanism comprises a drive cam having a cam lobe and wherein a second end of the needle carriage comprises a cam follower portion for engaging the cam lobe and being driven thereby.

14. A lancet assembly as claimed in claim 9 wherein the needle carriage has an elongate guided surface extending generally axially between the first and second ends of the needle carriage and wherein the guide element includes a guide surface for engaging and guiding the guided surface of the needle carriage.

15. A lancet assembly as claimed in claim 14 wherein the guide element within the housing and the guided surface of the needle carriage define a sliding dovetail connection.
